(19) United States
(12) Patent Application Publication
(10) Pub. No.: US 2009/0228000 A1
(43) Pub. Date: Sep. 10, 2009

(54) Disposable Apparatus for Patient Grounding

(76) Inventor: Michael Cao, St. Petersburg, FL (US)

Correspondence Address: Gray Robinson;ATTN: STEFAN V. STEIN/ IP DEPT., 201 N. Franklin Street, Suite 2200, Post Office Box 3324, TAMPA, FL, 33601-3324, US

(21) Appl. No.: 12/042,556

(22) Filed: Mar. 5, 2008

(51) Int. Cl. A61B 18/14

(52) U.S. Cl. 606/32

(57) ABSTRACT

An application for a grounding pad for use in electrosurgical procedures includes a base with a ground loop connector molded on a side edge of the base. A conductive coating covers at least part of a top surface of the base and continuing into the ground loop connector providing an electrical connection to a ground loop plug inserted into the ground loop connector. A pad retainer wall rises from the top surface of the base for holding a pad in place.

### FIELD OF THE INVENTION

This invention relates to the field of patient grounding and more particularly to an apparatus for grounding a patient during electrosurgical procedures.

### BACKGROUND OF THE INVENTION

The present invention relates to an improved grounding pad for use in electrosurgical procedures.

Electrosurgical procedures are in widespread use. For example, one such procedure uses electricity to reduce or eliminate hemorrhoids. Commonly, devices used in such procedures have two conductive probes, each carrying a different polarity of electric current. Thereby, electric current exits one probe conductor; passes through the affected tissue; and returns via the other probe conductor. This system works fine so long as both probes are in contact with the affected tissue. When only one probe conductor is in contact with the affected tissue, electric current from that probe passes through the patient's body and exits wherever a conduction path back to the electrosurgical device is available. This often causes discomfort from shocks and, in extreme cases, minor burns where the patient's skin contacts the path back to the electrosurgical device.

To reduce the discomfort and potential for burns, electrosurgical devices include a grounding facility to safely ground the patient to the electrosurgical device. The grounding facility is often placed against the patient's skin near the area where the electrosurgical device is to be used so as to prevent electrical current from flowing through any sensitive organs such as the heart.

Prior grounding devices include straps or pads that are connected to the ground potential of the electrosurgical device. The grounding pads of the prior art include a wire mesh connected to a ground cable that, in turn, is connected to the electrosurgical device. To prevent the wire mesh from cutting or scraping the patient, a cloth barrier is placed between the patient and the wire mesh. Since this cloth barrier presents an insulator to electrical current, the cloth barrier is moistened with a conductive solution (saline). In practice, due to costs, such grounding pads are often reused. Several issues exist with such grounding pads. The first is corrosion from the salt in the saline solution. This leads to the use of stainless steel, making the wire mess more expensive. Next, the wire mesh needs a connection to a lead wire that connects to the electrosurgical device. This connection is either a captured connection (e.g., the lead wire is soldered to the wire mesh) or includes a connector such as a banana plug and jack, in which case, the banana jack must be soldered to the mesh. Solder connections often fail, especially under environmental conditions where salt is present. The exposure to saline often causes an interment connection between the ground pad and the lead wire/connector with successive uses of the ground pad. Failure of the connection can result in shock and/or minor burns.

What is needed is an apparatus for patient grounding that is low cost making it disposable, reliable and less apt to cut or scratch the patient.

### SUMMARY OF THE INVENTION

One object of the present invention is the provision of an improved grounding pad which permits simplified usage during electrosurgical procedures.

Another object of the present invention is the provision of an improved grounding pad that provides improved contact with the patient's skin.

Another object of the present invention is the provision of an improved grounding pad which permits low cost manufacture, thereby permitting disposal after use.

Other and further objects of the invention will be obvious upon an understanding of the illustrative embodiment about to be described, or will be indicated in the appended claims, and various advantages not referred to herein will occur to one skilled in the art upon employment of the invention in practice.

In one embodiment, a grounding system for use in electrosurgical procedures is disclosed including a base and a ground loop connector molded on a side edge of the base. A conductive coating covers at least part of a top surface of the base. The conductive coating continues into the ground loop connector providing an electrical connection to a ground loop plug inserted into the ground loop connector.

In another embodiment, method of grounding a patient during an electrosurgical procedure is disclosed including providing a grounding pad that includes a base and a ground loop connector molded on a side edge of the base. The ground pad has a conductive coating covering at least part of a top surface of the base and continuing into the ground loop connector providing an electrical connection to a ground loop plug inserted into the ground loop connector. The base also has a pad retainer wall rising from its top surface and a pad sized to fit in the pad retainer wall. The pad is placed on the top surface of the base. The method continues with connecting a ground loop plug to the ground loop connector, the ground loop plug electrically connected to an electrosurgical apparatus through a ground loop wire then moistening the pad with a conductive solution. The grounding pad is placed in contact a patient and the electrosurgical procedure is performed.

In another embodiment, a grounding pad for use in electrosurgical procedures is disclosed including a base with a ground loop connector molded on a side edge of the base. A conductive coating covers at least part of a top surface of the base and continuing into the ground loop connector providing an electrical connection to a ground loop plug inserted into the ground loop connector. A pad retainer wall rises from the top surface of the base for holding a pad in place.

### BRIEF DESCRIPTION OF THE DRAWINGS

The invention can be best understood by those having ordinary skill in the art by reference to the following detailed description when considered in conjunction with the accompanying drawings in which:

FIG. 1 illustrates a perspective view of a grounding pad of the present invention.

FIG. 2 illustrates a side plan view of the present invention.

FIG. 3 illustrates a second side plan view of the present invention.

FIG. 4 illustrates an exploded view of the present invention.

FIG. 5 illustrates a magnified view of the connection jack of the present invention.

FIG. 6 illustrates a bottom perspective view of the present invention.

### DETAILED DESCRIPTION OF THE INVENTION

Reference will now be made in detail to the presently preferred embodiments of the invention, examples of which are illustrated in the accompanying drawings. Throughout the following detailed description, the same reference numerals refer to the same elements in all figures.

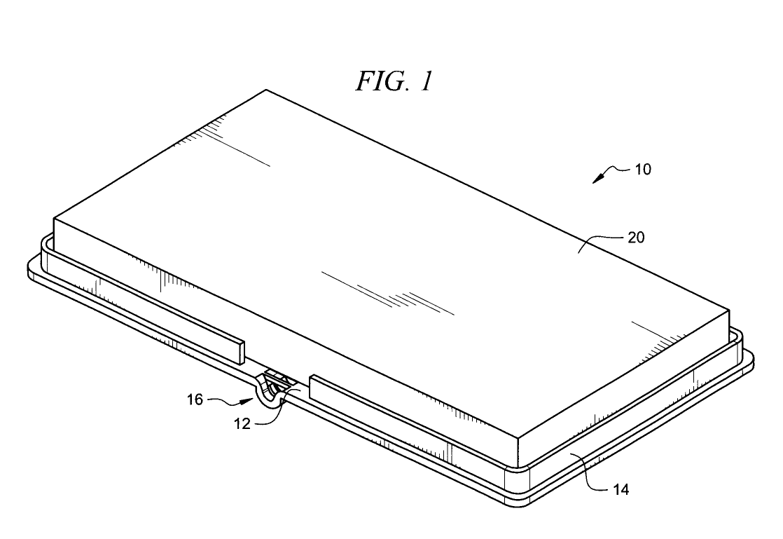

Referring to FIG. 1, a perspective view of a grounding pad of the present invention is shown. The grounding pad 10 has a base 12 and a pad retainer wall 14. In some embodiments, the pad retainer wall 14 is fabricated separately from the base 12 and affixed to the base 12 as known in the industry. In other embodiments, the pad retainer wall 14 is over-molded onto the base 12. A pad 20 provides a soft, cushioned interface with the patient. Although the pad 20 is any absorbent material known in the industry, it is preferred that the pad 20 be a cellulose sponge.

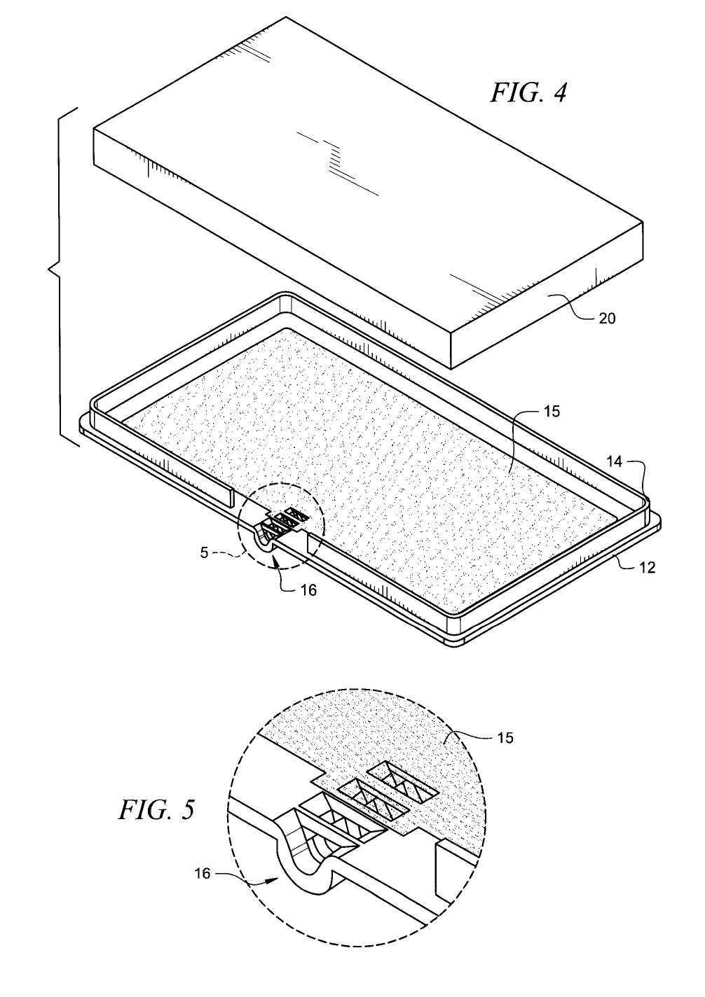

In the preferred embodiment, a ground loop connector 16 provides for electrical connection to the electrosurgical apparatus. In other embodiments (not shown), a ground loop wire is directly connected to the ground pad 10. As will be shown, the base 12 is coated with conductive coating 15 (see FIG. 4) and the conductive coating 15 continues into the ground loop connector 16, thereby providing a path for conduction of electricity from a ground loop wire, through the ground loop connector 16, across the base 12 and into the pad 20. So that the electricity flows through to the patient, the pad is moistened with a conductive solution such as saline.

During an electrosurgical operation such as an electrosurgical operation for the treatment of hemorrhoids, the grounding pad 10 is fitted with a pad 20, the pad 20 is moistened with a conductive solution (e.g., saline), the ground loop connector 16 is connected to the electrosurgical device and the ground pad 10 is placed such that the pad 20 contacts the patient's skin. For hemorrhoid treatment, the ground pad 10 is preferably placed beneath the buttocks of the patient. Stray electrical current passes from the electrosurgical device, through the patient, through the pad 20, through the conductive coating 15, through the ground loop connector 16, through a ground loop cable and into the electrosurgical apparatus.

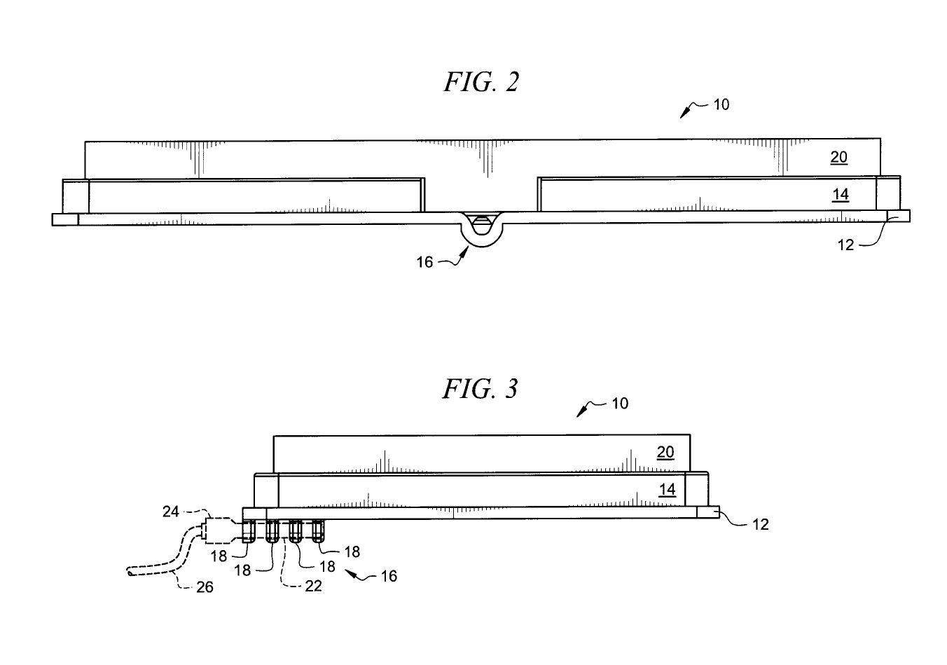

Referring to FIG. 2, a side plan view of the present invention is shown. The grounding pad 10 has a base 12 and a pad retainer wall 14. The opening of the ground loop connector 16 is visible from this angle. It is preferred that the pad 20 be thicker than the height of the pad retainer wall 14 to provide comfort to the patient.

Referring to FIG. 3, a second side plan view of the present invention is shown. The grounding pad 10 has a base 12 and a pad retainer wall 14. The loops 18 of the ground loop connector 16 are visible from this angle. Although four loops 18 are shown, any number of loops 18 are anticipated. A plug 24 is shown with its metal contacts 22 inserted into the loops 18 of the ground loop connector 16 and a ground loop wire connects the plug 22/24 with the electrosurgical apparatus (not shown). Again, it is preferred that the pad 20 be thicker than the height of the pad retainer wall 14 to provide comfort to the patient.

Referring to FIGS. 4 and 5, an exploded view of the present invention is shown. The grounding pad 10 has a base 12 and a pad retainer wall 14. An area on the top side of the base, within the pad retainer walls 14, is coated with a conductive layer 15. In the preferred embodiment, the conductive layer 15 is conductive paint such as paints that include gold in their list of ingredients. In other embodiments, the conductive layer 15 is a layer of metal formed by electroplating as is known in the industry.

The conductive layer 15 continues into the recesses of the ground loop connector 16, coating at least one inner surface of the rings 18, thereby completing the circuit between the ground loop plug 22/24 and the conductive layer 15 on the surface of the grounding pad 10.

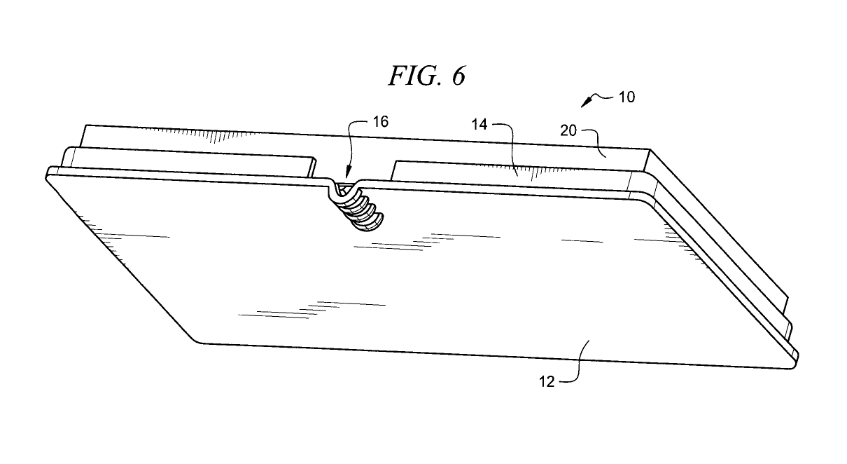

Referring to FIG. 6, a bottom perspective view of the present invention is shown. The grounding pad 10 has a base 12 and a pad retainer wall 14. Although the bottom surface of the base 12 is shown relatively planar except for the ground loop connector 16, in some embodiments, the bottom surface of the base 12 has standoffs, etc., to reduce play when placed on a hard surface such as an operating table.

Equivalent elements can be substituted for the ones set forth above such that they perform in substantially the same manner in substantially the same way for achieving substantially the same result.

It is believed that the system and method of the present invention and many of its attendant advantages will be understood by the foregoing description. It is also believed that it will be apparent that various changes may be made in the form, construction and arrangement of the components thereof without departing from the scope and spirit of the invention or without sacrificing all of its material advantages. The form herein before described being merely exemplary and explanatory embodiment thereof. It is the intention of the following claims to encompass and include such changes.

What is claimed is:

1. A grounding system for use in electrosurgical procedures, the grounding system comprising:
a base;
a ground loop connector molded on a side edge of the base; and
a conductive coating covering at least part of a top surface of the base, the conductive coating continuing into the ground loop connector providing an electrical connection to the ground loop connector.

2. The grounding system of claim 1, wherein the conductive coating is a conductive paint.

3. The grounding system of claim 1, further comprising a pad retainer wall, the pad retainer wall rising from a peripheral area of the top surface of the base.

4. The grounding system of claim 1, further comprising a pad, the pad sized to fit within the pad retainer wall.

5. The grounding system of claim 4, wherein the pad is thicker than the height of the pad retainer wall.

6. The grounding system of claim 4, wherein the pad is a cellulose sponge.

7. A method of grounding a patient during an electrosurgical procedure, the method comprising:
providing a grounding pad, the grounding pad comprising:
a base;
a ground loop connector molded on a side edge of the base;
a conductive coating covering at least part of a top surface of the base, the conductive coating continuing into the ground loop connector providing an electrical connection to the ground loop connector;
a pad retainer wall, the pad retainer wall rising from a peripheral area of the top surface of the base; and
a pad, the pad sized to fit within the pad retainer wall;
connecting a ground loop plug to the ground loop connector, the ground loop plug electrically connected to an electrosurgical apparatus through a ground loop wire;
moistening the pad with a conductive solution;
placing the grounding pad in contact with a patient; and
performing the electrosurgical procedure.

8. The method of claim 7, wherein the conductive solution is saline.

9. The method of claim 7, wherein the electrosurgical device is an electrosurgical device for the treatment of hemorrhoids and the step of placing the grounding pad in contact with the patient includes placing the grounding pad beneath the buttocks of the patient.

10. The method of claim 7, wherein the pad is a cellulose sponge.

11. The method of claim 7, wherein the conductive coating is conductive paint.

12. The method of claim 11, wherein the conductive paint includes gold.

13. A grounding pad for use in electrosurgical procedures, the grounding pad comprising:
a base;
a ground loop connector molded on a side edge of the base;
a conductive coating covering at least part of a top surface of the base, the conductive coating continuing into the ground loop connector providing an electrical connection to the ground loop connector; and
a pad retainer wall, the pad retainer wall rising from a periphery of the top surface of the base.

14. The grounding pad of claim 13, wherein the base, the ground loop connector and the pad retainer is made of plastic.

15. The grounding pad of claim 13, wherein the conductive coating is conductive paint.

16. The grounding pad of claim 15, wherein the conductive paint includes gold.

17. The grounding pad of claim 13, further comprising a pad the pad sized to fit within the pad retainer wall, the pad is thicker than the height of the pad retainer wall.

18. The grounding pad of claim 17, wherein the pad is a cellulose sponge.
